(19) United States
(12) Patent Application Publication
(10) Pub. No.: US 2026/0083731 A1
(43) Pub. Date: Mar. 26, 2026

(54) METHODS OF TREATING UVEAL MELANOMA WITH A PKC INHIBITOR

(71) Applicant: IDEAYA BIOSCIENCES, INC., South San Francisco, CA (US)

(72) Inventors: MATTHEW ANTHONY MAURER, South San Francisco, CA (US); MICHAEL GABRIEL O'QUIGLEY, South San Francisco, CA (US); JULIE HAMBLETON, South San Francisco, CA (US)

(21) Appl. No.: 19/106,906

(22) PCT Filed: Aug. 25, 2023
(86) PCT No.: PCT/US2023/072953

§ 371 Date: Feb. 26, 2025

Related U.S. Application Data
(60) Provisional application No. 63/373,688, filed on Aug. 26, 2022.
(60) Provisional application No. 63/375,141, filed on Sep. 9, 2022.
(60) Provisional application No. 63/386,851, filed on Dec. 9, 2022.
(60) Provisional application No. 63/497,362, filed on Apr. 20, 2023.
(60) Provisional application No. 63/509,256, filed on Jun. 20, 2023.

(51) Int. Cl. A61K 31/497; A61N 5/10; A61P 35/00

(52) U.S. Cl. CPC A61K 31/497; A61N 5/1017; A61P 35/00; A61N 2005/1024; A61N 2005/1094; A61N 2005/1096; A61N 2005/1098

(57) ABSTRACT

Provided herein is a neoadjuvant therapy and/or adjuvant therapy for the treatment of uveal melanoma, inter alia, ocular tumors in uveal melanoma to reduce the incidence of metastasis and/or enucleation and/or preserving eye function.

### RELATED APPLICATIONS

This application claims priority to U.S. Provisional Application No. 63/373,688, filed on Aug. 26, 2022; U.S. Provisional Application No. 63/375,141, filed on Sep. 9, 2022; U.S. Provisional Application No. 63/386,851, filed on Dec. 9, 2022; U.S. Provisional Application No. 63/497,362, filed on Apr. 20, 2023; and U.S. Provisional Application No. 63/509,256, filed on Jun. 20, 2023. The content of each application is incorporated by reference in their entirety.

### BACKGROUND

Uveal melanoma (UM) is the most common primary intraocular malignant tumor in adults. Certain protein kinase inhibitors are described in International Publ. Nos. WO 02/38561 and WO 2008/106692. One protein kinase C (PKC) inhibitor, sotrastaurin, has been shown to have activity against certain PKC isototypes and has only recently been shown to selectively inhibit the growth of uveal melanoma cells harboring GNAQ mutations by targeting PKC/ERK1/2 and PKC/NF-xB pathways (see X. Wu, et al., Mol. Cancer Ther., Vol. 11, pages 1905-1914, 2012). However, there still remains an unmet need to provide next generation PKC inhibitors for treating uveal melanoma that have improved efficacy at lower dosage amounts to achieve tumor regression, improved potency, hERG activity, absorption, gastrointestinal tolerance and kinase selectivity. PCT application no. PCT/IB2015/055951 (WO 2016/020864) discloses a number of potent and selective PKC inhibitors.

Management of primary UM varies from observation to removal, or enucleation, of the eye and its immediate surrounding structures, depending on tumor features. Most patients with UM are currently treated with radiotherapy (e.g., plaque brachytherapy or stereotactic radiosurgery), which preserves the eye. Both of these exemplary approaches, however, are associated with similar rates of development of subsequent metastatic disease. Intraocular tumors that are too large (e.g., greater than 10 mm thick and/or 16 mm wide) are not generally amenable to radiotherapy due to low rates of local control and/or high rates of vision and eye pain. Enucleation is typically necessary for these larger tumors, but significantly impacts patient quality of life via immediately reduced vision, decreased depth perception, diminished social functioning and unsatisfactory cosmesis. There is currently no treatment available to shrink large intraocular UMs, allowing for eye preservation or retention of visual function, wholly or partially. Additionally, it is desirable to reduce the need for enucleations, and potentially decrease metastatic relapse as described herein.

Provided herein is a PKC targeted therapy to address some of the issues discussed herein.

### SUMMARY

Provided herein is a neoadjuvant therapy comprising administering a PKC inhibitor. Also provided herein is a primary therapy for treatment of uveal melanoma comprising administering a PKC inhibitor. The neoadjuvant therapy and the primary therapy are, in each case, useful for the treatment of uveal melanoma, including ocular tumors. In one aspect, the ocular tumors are intraocular tumors. In another aspect, a PKC inhibitor is also useful as an adjuvant therapy. A neoadjuvant therapy can be administered in advance of or during an interventional procedure, which can be a primary interventional procedure. An adjuvant therapy can be used during or after an interventional procedure, which can be a primary interventional procedure. In each such case, a (primary) interventional procedure can include, for example, irradiation of the ocular tumor or surgical intervention (e.g., resection or removal) of the ocular tumor.

Thus, in one aspect, provided herein is a method of treating uveal melanoma in a patient having an ocular tumor, the method comprising a) administering a PKC inhibitor to the patient as a neoadjuvant therapy; b) reducing the size of the ocular tumor by at least 10%; and c) treating the reduced-size ocular tumor with an interventional procedure.

In another aspect, provided herein is a method of treating a patient having uveal melanoma with an ocular tumor with a size indicated for an interventional procedure comprising irradiating the ocular tumor, the method comprising a) administering a PKC inhibitor to the patient as a neoadjuvant therapy; b) reducing the size of the ocular tumor by at least 10%; and c) treating the reduced-size ocular tumor with an interventional procedure other than enucleation, including for example with an interventional procedure comprising irradiation.

In another aspect, provided herein is a method of treating a patient having uveal melanoma with an ocular tumor with a size indicated for an interventional procedure comprising enucleation of the eye which is the situs of the ocular tumor, the method comprising a) administering a PKC inhibitor to the patient as a neoadjuvant therapy; b) reducing the size of the ocular tumor by at least 10%; and c) treating the reduced-size ocular tumor with an interventional procedure other than enucleation. In each of these such aforementioned aspects of the invention, the method of treatment can comprise administering a PKC inhibitor to the patient as a neoadjuvant therapy whereby the size of the ocular tumor is reduced by at least 10%.

In various other aspects, as described in more detail below, the method of treatment of uveal melanoma can comprise administering a PKC inhibitor to the patient as a neoadjuvant therapy to reduce the size of an ocular tumor to enable an alternative treatment paradigm. For example, and without limitation except as explicitly claimed, neoadjuvant treatment with a PKC inhibitor can reduce a large size ocular tumor to either a medium size ocular tumor or a small size ocular tumor, to enable an interventional procedure other than enucleation (e.g., for a medium size ocular tumor) or to enable an observational period (e.g., for a small size ocular tumor) prior to or in lieu of an interventional procedure. As another example, and without limitation except as explicitly claimed, neoadjuvant treatment with a PKC inhibitor can reduce a medium size ocular tumor to be a small size ocular tumor, to enable an interventional procedure other than enucleation, such as an alternative radiotherapy approach than otherwise would have been applicable for the original medium size ocular tumor or to enable an to enable an observational period prior to or in lieu of an interventional procedure.

In each of such aspects of the invention, an ocular tumor size can be defined by a set of dimensions known in the art. Such dimensions can include for example, a basal diameter, typically measured a longest basal diameter (LBD) of the ocular tumor and/or an apical height. Another feature of an ocular tumor that can impact selection of an interventional procedure is the relative proximity of the location or situs of the ocular tumor to the ocular nerve. Accordingly, various such aspects of the invention are described herein, including in connection with certain such dimensions as defined by guidelines issued by regulatory agencies or other governmental or private authoritative bodies, such as the National Comprehensive Cancer Network (NCCN) Guidelines Version 2.2022 (Uveal Melanoma), National Cancer Institute (NCI) clinical practice guidelines, among others known in the art, and/or by standard of care practices developed by ocular oncologists or ocular surgeons informed of such guidelines and practicing in the field of treating uveal melanoma.

In another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising a) administering a PKC inhibitor to the patient as a neoadjuvant therapy; b) reducing the size of the ocular tumor to dimensions of less than 5 mm LBD and less than 3 mm apical height; and optionally c) treating the reduced-size ocular tumor with an interventional procedure. Without limitation except as explicitly claimed, such aspect can reflect reducing a large size ocular tumor, e.g., with dimensions as defined as in (i) or (ii), or a medium size tumor, e.g., with dimensions as defined as in (iii), in each case to a small size ocular tumor. Such a small size ocular tumor may be monitored during an observational period prior to or in lieu of an interventional procedure.

In yet another aspect, provided herein is a method of treating uveal melanoma in a patient having an ocular tumor with a size defined by a set of dimensions comprising (i) greater than 16 mm LBD and any apical height, or (ii) any LBD and greater than 8 mm apical height, the method comprising a) administering a PKC inhibitor to the patient as a neoadjuvant therapy; b) reducing the size of the ocular tumor to dimensions of less than 16 mm LBD and less than 8 mm apical height; and optionally c) treating the reduced-size ocular tumor with an interventional procedure. Without limitation except as explicitly claimed, such aspect can reflect reducing a large size ocular tumor, e.g., with dimensions as defined as in (i) or (ii), in each case to a medium size ocular tumor or a small size ocular tumor.

In the immediately preceding aspect, for example, for a patient having an ocular tumor with a size defined by a set of dimensions comprising (i) greater than 16 mm LBD and any apical height or (ii) any LBD and greater than 8 mm apical height, the method of treating uveal melanoma can comprise a) administering a PKC inhibitor to the patient as a neoadjuvant therapy; b) reducing the size of the ocular tumor to dimensions of less than 16 mm LBD and 3-8 mm apical height; and c) treating the reduced-size ocular tumor with an interventional procedure. Without limitation except as explicitly claimed, this example can reflect reducing a large size ocular tumor, e.g., with dimensions as defined as in (i) or (ii), in each case to a medium size ocular tumor.

In still another aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) any LBD, greater than 8 mm apical height, and the ocular tumor is located close to, including proximal to or adjacent, the optic nerve, or (iv) less than or equal to 19 mm LBD and 2.5-10 mm apical height, the method comprising a) administering a PKC inhibitor as a neoadjuvant therapy; b) reducing the size of the ocular tumor to dimensions of 5-19 mm LBD or less than 2.5 mm apical height; and c) treating the reduced-size ocular tumor with an interventional procedure. Preferably, step (b) of this aspect comprises (b) reducing the size of the ocular tumor to dimensions of 5-19 mm LBD and less than 2.5 mm apical height. Without limitation except as explicitly claimed, this example can reflect reducing a large size ocular tumor, e.g., with dimensions as defined as in (i), (ii), (iii) or (iv), in each case to a medium size ocular tumor or a small size ocular tumor.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the ocular tumor is located close to, including proximal to or adjacent, the optic nerve, the method comprising a) administering a PKC inhibitor to the patient as a neoadjuvant therapy; b) reducing the size of the ocular tumor to dimensions of less than or equal to 19 mm LBD and 2.5-10 mm apical height; and c) treating the reduced-size ocular tumor with an interventional procedure. Without limitation except as explicitly claimed, this example can reflect reducing a large size ocular tumor, e.g., with dimensions as defined as in (i), (ii), (iii), in each case to a medium size ocular tumor or a small size ocular tumor.

### DETAILED DESCRIPTION

Provided herein is a neoadjuvant therapy comprising a PKC inhibitor, or a pharmaceutically acceptable salt thereof. This therapy is useful for the treatment of uveal melanoma, including ocular tumors. This therapy is also useful as an adjuvant therapy.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, including ±5%, +1%, and +0.1% from the specified value, as such variations are appropriate to perform the disclosed methods. For example, a dose of about 300 mg may be understood to mean that the dose may vary between 270 mg and 330 mg.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond, e.g., propenyl, butenyl, and the like.

“Alkynyl” means a linear monovalent hydrocarbon radical of two to six carbon atoms or a branched monovalent hydrocarbon radical of three to six carbon atoms containing a triple bond, e.g., ethynyl, propynyl, butynyl, and the like.

“Alkoxy” means a —OR radical where R is alkyl as defined above, e.g., methoxy, ethoxy, propoxy, or 2-propoxy, n-, iso-, or tert-butoxy, and the like.

“Aryl” means a monovalent monocyclic or bicyclic aromatic hydrocarbon radical of 6 to 10 ring atoms e.g., phenyl or naphthyl.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to as fluoroalkoxy.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heterocyclyl” or “heterocycloalkyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When heterocyclyl contains at least one nitrogen atom, it may be referred to herein as heterocycloamino.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a dose range of “200 mg to about 600 mg” should be interpreted to include not only the explicitly recited concentration of about 200 mg to about 600 mg, but also include individual dosage (e.g., 250 mg, 400 mg, 550 mg) and the sub-ranges (e.g., 250 mg to 450 mg) within the indicated range. To further illustrate, a tumor size reduction of “30%-50%” should be interpreted to include not only the explicitly recited concentration of about 30% to about 50%, but also include individual percentages (e.g., 35%, 40%, 50%) and the sub-ranges (e.g., 35%-45%) within the indicated range. The term “about” can include ±1%, ±2%, ±3%, ±4%, ±5%, ±6%, ±7%, ±8%, ±9%, or ±10%, of the numerical value(s) being modified. In addition, the phrase “about ‘x’ to ‘y’” includes “about ‘x’ to about ‘y’”.

As used herein, the term “free base equivalent” refers to the amount of the active agent (e.g., Compound 1) present in the active agent or pharmaceutically acceptable salt thereof. Stated alternatively, the term “free base equivalent” means either an amount of Compound 1 free base, or the equivalent amount of Compound 1 free base that is provided by a salt of said compound.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “First-Line MUM patients” refers to patients that were not given prior systemic treatment in the metastatic setting including no prior chemo-embolization, no radiation to the metastatic sites or ablation to the liver lesions.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The dose amounts (for Compound 1) are expressed as free base equivalent amounts, unless indicated otherwise.

An “oral dosage form” includes a unit dosage form prescribed or intended for oral administration.

### Methods of Treatment

In an aspect, provided herein is a method of treating uveal melanoma in a patient having an ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure.

In an embodiment, the ocular tumor is defined by a set of dimensions comprising (i) greater than or equal to 5 mm largest basal diameter (LBD) and any apical height, or (ii) any LBD and greater than 3 mm apical height. In another embodiment, the ocular tumor is defined by being greater than or equal to 5 mm largest basal diameter (LBD) and any apical height. In yet another embodiment, the ocular tumor is defined by being any LBD and greater than 3 mm apical height.

In an aspect, provided herein is a method of treating a patient having uveal melanoma with an ocular tumor with a size indicated for an interventional procedure comprising irradiating the ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

In another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor to the patient as a neoadjuvant therapy;
b) reducing the size of the ocular tumor to dimensions of less than 5 mm LBD and less than 3 mm apical height; and
c) treating the reduced-size ocular tumor with an interventional procedure.

In yet another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor to the patient; and
b) reducing the size of the ocular tumor defined by a set of dimensions having less than 5 mm LBD and less than 3 mm apical height. In an embodiment, the method further comprising treating the reduced-size ocular tumor with an interventional procedure.

In yet another aspect, provided herein is a method of treating uveal melanoma in a patient having an ocular tumor with a size defined by a set of dimensions comprising (i) greater than 16 mm LBD and any apical height, or (ii) any LBD and greater than 8 mm apical height, the method comprising
a) administering a PKC inhibitor to the patient as a neoadjuvant therapy;
b) reducing the size of the ocular tumor to dimensions of less than 16 mm LBD and less than 8 mm apical height; and
c) treating the reduced-size ocular tumor with an interventional procedure. In an embodiment, the step (b) is reducing the size of the ocular tumor to dimensions of less than 16 mm LBD and 3-8 mm apical height.

In still another aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor to the patient as a neoadjuvant therapy;
b) reducing the size of the ocular tumor defined by a set of dimensions having 5-19 mm LBD or less than 2.5 mm apical height; and
c) treating the reduced-size ocular tumor with an interventional procedure. In an embodiment, the step (b) is reducing the size of the ocular tumor to dimensions of 5-19 mm LBD and less than 2.5 mm apical height.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor to the patient as a neoadjuvant therapy; and
b) reducing the size of the ocular tumor defined by a set of dimensions having 5-19 mm LBD and less than 2.5 mm apical height. In an embodiment, the method further comprising treating the reduced-size ocular tumor with an interventional procedure.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
a) administering a PKC inhibitor to the patient as a neoadjuvant therapy;
b) reducing the size of the ocular tumor to dimensions of less than or equal to 19 mm LBD and 2.5-10 mm apical height; and
c) treating the reduced-size ocular tumor with an interventional procedure.

In another aspect, provided herein is a method of treating a patient having uveal melanoma with an ocular tumor with a size indicated for an interventional procedure comprising enucleation of the eye, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

In another aspect, provided herein is a method of treating a patient having uveal melanoma with an ocular tumor with a size indicated for an interventional procedure comprising enucleation of the eye, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

In yet another aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor by at least 10%; and
d) irradiating the reduced-size ocular tumor with a second therapeutically effective dose of radiation less than the first therapeutically effective dose of radiation.

In still another aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor; and
d) irradiating the reduced-size ocular tumor with a second therapeutically effective dose of radiation at least 10% less than the first therapeutically effective dose of radiation.

In an aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy to reduce the size of the ocular tumor by at least 10%; and
c) irradiating the reduced-size ocular tumor with a second therapeutically effective dose of radiation less than the first therapeutically effective dose of radiation.

In another aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy to reduce the size of the ocular tumor; and
c) irradiating the reduced-size ocular tumor with a second therapeutically effective dose of radiation at least 10% less than the first therapeutically effective dose of radiation.

In yet another aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy over a period of time sufficient to reduce the size of the ocular tumor by at least 10%; and
c) irradiating the reduced-size ocular tumor with a second therapeutically effective dose of radiation less than the first therapeutically effective dose of radiation.

In still another aspect, provided herein is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy over a period of time sufficient to reduce the size of the ocular tumor; and
c) irradiating the reduced-size ocular tumor with a second therapeutically effective dose of radiation at least 10% less than the first therapeutically effective dose of radiation.

In an aspect, provided herein is a method of treating a patient having uveal melanoma with an ocular tumor of a size indicated for an interventional procedure comprising irradiating the ocular tumor, the method comprising:
a) administering a PKC inhibitor to the patient as neoadjuvant therapy;
b) reducing the size of the ocular tumor by at least 10%; and
c) irradiating the reduced-size ocular tumor with a therapeutically effective dose of radiation, wherein the therapeutically effective dose of radiation is less than a dose of radiation required to irradiate the ocular tumor in the absence of neoadjuvant therapy.

In an embodiment, the interventional procedure comprises local surgical resection of the tumor, irradiating the tumor, or any combination thereof. In another embodiment, the interventional procedure is local surgical resection of the tumor. In yet another embodiment, the interventional procedure is irradiating the tumor.

In some embodiments, the PKC inhibitor is a compound of Formula I:

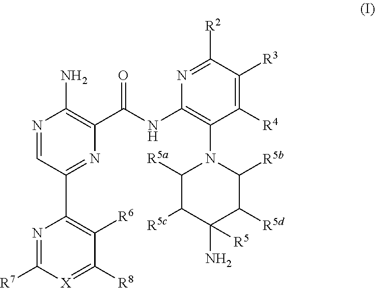

or a pharmaceutically acceptable salt thereof,
wherein:
X is N or CR;
R, R², R³ and R⁴ are each independently H, ²H, halo, OH, C₁₋₃ alkoxy, C₁₋₃haloalkyl or C₁₋₃ alkyl, said C₁₋₃ alkyl optionally substituted with OH, C₁₋₃ alkoxy or C₁₋₃ haloalkoxy;
R⁵ is independently H, ²H, or C₁₋₃ alkyl, said C₁₋₃ alkyl optionally substituted with F, OH, C₁₋₃ alkoxy, or C₁₋₃ haloalkoxy;
R^{5a} and R^{5b} are each independently H, ²H, or C₁₋₃ alkyl, said C₁₋₃ alkyl optionally substituted with F, OH or C₁₋₃ alkoxy, or R^{5a} and R^{5b} are joined together forming a methylene or ethylene bridging group;
R^{5c} and R^{5d} are each independently H, ²H, F, OH, C₁₋₃ alkoxy, or C₁₋₃ alkyl, said C₁₋₃ alkyl optionally substituted with F, OH or C₁₋₃ alkoxy, or R^{5c} and R^{5d} are joined together forming a methylene, ethylene or —CH₂—O— bridging group; and
R⁶, R⁷ and R⁸ are each independently selected from H, ²H, halo, C₁₋₃alkyl, C₁₋₃ haloalkyl, C₁₋₃ alkoxy, C₁₋₃ haloalkoxy, C₃₋₇ cycloalkyl and 4-7 membered heterocycloalkyl having 1 to 3 heteroatoms selected from N, O and S, said C₁₋₃ alkyl optionally substituted with F, OH, C₁₋₃ alkoxy or C₁₋₃ haloalkoxy.

In another embodiment, the PKC inhibitor is Compound 1:

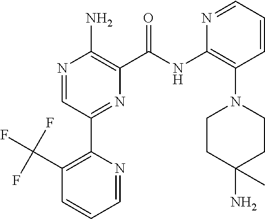 [SMILES: CC1(N)CCN(c2cccnc2NC(=O)c2nc(-c3ncccc3C(F)(F)F)cnc2N)CC1]

or a pharmaceutically acceptable salt thereof.

In another embodiment, the PKC inhibitor is Compound 1.

In another embodiment, the method further comprises continuing administering a PKC inhibitor to the patient as an adjuvant therapy during the period of the interventional procedure.

In yet another embodiment, the method further comprises continuing administering a PKC inhibitor to the patient as an adjuvant therapy after the completion of the interventional procedure.

In still another embodiment, the PKC inhibitor is administered orally.

In an embodiment, the method further comprises preserving the eye globe which had the ocular tumor.

In another embodiment, the method further comprises preserving a function of an eye which had the ocular tumor. In yet another embodiment, preserving the function of the eye which had the ocular tumor comprises at least partially preserving the eye function.

In still another embodiment, the method further comprises preventing irreversible vision loss. In an embodiment, the method further comprises preserving visual symptoms. In another embodiment, the method further comprises preserving depth perception. In yet another embodiment, the method further comprises preserving vision for greater than at least 1 year. In still another embodiment, the method further comprises preventing metastasis. In an embodiment, the method further comprises delaying onset of metastasis.

In another embodiment, the ocular tumor is spatially separated from the optic nerve.

In yet another embodiment, the ocular tumor, prior to administering the neoadjuvant therapy, has a size from 5 mm to 19 mm LBD and less than 2.5 mm apical height. In still another embodiment, the ocular tumor, prior to administering the neoadjuvant therapy, has a size less than or equal to 19 mm LBD and 2.5-10 mm apical height.

In another embodiment, the ocular tumor, prior to administering the neoadjuvant therapy, has a size greater than 19 mm LBD. In yet another embodiment, the ocular tumor, prior to administering the neoadjuvant therapy, has a size greater than 10 mm apical height and any size LBD. In still another embodiment, the ocular tumor, prior to administering the neoadjuvant therapy, has a size greater than 8 mm apical height and the tumor is located close to the optic nerve.

In an embodiment, the ocular tumor, prior to administering the neoadjuvant therapy, has a size of greater than 6 mm and less than 12 mm apical height, and not greater than 16 mm LBD. In another embodiment, the ocular tumor, prior to administering the neoadjuvant therapy, has a size of greater than 3 mm and less than 8 mm apical height, and not greater than 16 mm LBD. In yet another embodiment, the ocular tumor, prior to administering the neoadjuvant therapy, has a size of greater than 8 mm apical height, and greater than 16 mm LBD. In still another embodiment, the ocular tumor, prior to administering the neoadjuvant therapy, is not greater than 16 mm LBD. In an embodiment, the size of the ocular tumor, prior to administering the neoadjuvant therapy, is greater than 10 mm in apical height.

In another embodiment, the size of the ocular tumor after administering the neoadjuvant therapy but prior to the interventional procedure is reduced by at least 20%. In another embodiment, the size of the ocular tumor after administering the neoadjuvant therapy but prior to the interventional procedure is reduced by at least 30%. In another embodiment, the size of the ocular tumor after administering the neoadjuvant therapy but prior to the interventional procedure is reduced by at least 40%. In yet another embodiment, the size of the ocular tumor after administering the neoadjuvant therapy but prior to the interventional procedure is reduced by at least 50%. In another embodiment, the size of the ocular tumor after administering the neoadjuvant therapy but prior to the interventional procedure is reduced by at least 10%, at least 15%, at least 20%, at least 25%, at least 30%, at least 35%, at least 40%, at least 45%, at least 50%, at least 60%, at least 65%, at least 70%, or at least 75%.

In another embodiment, the LBD of the ocular tumor after administering the neoadjuvant therapy but prior to the interventional procedure is reduced by at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm. In another embodiment, the LBD of the ocular tumor after administering the neoadjuvant therapy but prior to the interventional procedure is reduced by at least 1 mm, at least 2 mm, at least 3 mm, at least 4 mm, or at least 5 mm.

In another embodiment, the apical height of the ocular tumor after administering the neoadjuvant therapy but prior to the interventional procedure is reduced by at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm. In another embodiment, the apical height LBD of the ocular tumor after administering the neoadjuvant therapy but prior to the interventional procedure is reduced by at least 1 mm, at least 2 mm, at least 3 mm, at least 4 mm, or at least 5 mm.

In still another embodiment, the PKC inhibitor, or an equivalent dose of a pharmaceutically acceptable salt thereof, is administered at a dose of about 200 mg to 400 mg two times per day (BID). In still another embodiment, the PKC inhibitor, or an equivalent dose of a pharmaceutically acceptable salt thereof, is administered at a dose of about 300 mg two times per day (BID). In another embodiment, the PKC inhibitor, or an equivalent dose of a pharmaceutically acceptable salt thereof, is administered at a dose of about 400 mg to about 600 mg daily.

In yet another embodiment, the PKC inhibitor, or an equivalent dose of a pharmaceutically acceptable salt thereof is administered daily for at least 7 days, at least 14 days, at least 21, days, at least 28 days, at least 56 days, at least 84 days, at least, 112 days, at last 140 days, or at least 168 days.

In still another embodiment, the interventional procedure is selected from the group consisting of plaque brachytherapy (PBT), external beam radiotherapy (EBRT), stereotactic radiosurgery (SRS), and any combination thereof. In an embodiment, the interventional procedure is a globe preserving treatment (GPT). In still another embodiment, the interventional procedure is plaque brachytherapy (PBT). In still another embodiment, the interventional procedure is external beam radiotherapy (EBRT). In still another embodiment, the interventional procedure is stereotactic radiosurgery (SRS). In still another embodiment, the plaque brachytherapy (PBT) is Ruthenium plaque brachytherapy.

In another embodiment, the interventional procedure is a globe preserving treatment (GPT). In yet another embodiment, the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

In yet another embodiment, the therapeutically effective dose of radiation is determined based on the ocular tumor size, location, or a combination thereof.

In still another embodiment, the size and location of the ocular tumor is determined by CT scan, MRI, or a combination thereof. In another embodiment, the size or location of the ocular tumor is determined by CT scan, MRI, or a combination thereof. In another embodiment, the size and/or location of the ocular tumor is determined by ultrasound.

In an embodiment, the therapeutically effective dose of radiation is determined using Plaque Simulator. In another embodiment, the Plaque Simulator is version 6 (PS6).

In yet another embodiment, the method further comprises continuing administering a PKC inhibitor, or an equivalent dose of a pharmaceutically acceptable salt thereof, to the patient as an adjuvant therapy during the period of the irradiation. In still another embodiment, the method further comprises continuing administering a PKC inhibitor, or an equivalent dose of a pharmaceutically acceptable salt thereof, to the patient as an adjuvant therapy after the completion of the irradiation.

In an embodiment, determining a first therapeutically effective dose of radiation for treating the ocular tumor comprises determining a therapeutically effective dose of radiation to fovea before administering the PKC inhibitor, or an equivalent dose of a pharmaceutically acceptable salt thereof, to the patient. In yet another embodiment, irradiating the reduced-size ocular tumor with a second therapeutically effective dose of radiation comprises determining a therapeutically effective dose of radiation to fovea. In still another embodiment, irradiating the reduced-size ocular tumor with a therapeutically effective dose of radiation comprises determining a therapeutically effective dose of radiation to fovea.

Exemplary interventional procedures can be found, at least, in (i) Foti P V et al., Diagnostic methods and therapeutic options of uveal melanoma with emphasis on MR imaging-Part II: treatment indications and complications. Insights Imaging. 2021 Jun. 4; 12(1):67, and (ii) Rao Y J, et al., Patterns of care and survival outcomes after treatment for uveal melanoma in the post-coms era (2004-2013): a surveillance, epidemiology, and end results analysis. J Contemp Brachytherapy. 2017 October; 9(5):453-465, the contents of which are incorporated in their entirety.

In still another embodiment, the uveal melanoma is a solid tumor harboring GNAQ or GNA11 mutations. In an embodiment, the patient has additional non-ocular tumor. In an embodiment, the non-ocular tumor is metastatic uveal melanoma (MUM). In an embodiment, the ocular tumor is malignant. In another embodiment, the ocular tumor is not malignant. In another embodiment, the ocular tumor is intraocular tumor.

In another embodiment, Compound 1, or a pharmaceutically acceptable salt thereof, is administered at a dose of about 400 mg to about 600 mg daily of free base equivalent of Compound 1.

In yet another embodiment, Compound 1, or a pharmaceutically acceptable salt thereof, is administered at a dose of about 100 mg BID of free base equivalent of Compound 1. In yet another embodiment, Compound 1, or a pharmaceutically acceptable salt thereof, is administered at a dose of about 200 mg BID of free base equivalent of Compound 1. In still another embodiment, Compound 1, or a pharmaceutically acceptable salt thereof, is administered at a dose of about 300 mg BID of free base equivalent of Compound 1. In another embodiment, Compound 1, or a pharmaceutically acceptable salt thereof, is administered at a dose of about 400 mg BID of free base equivalent of Compound 1.

In an embodiment, the uveal melanoma is metastatic uveal melanoma. In another embodiment, the uveal melanoma is a solid tumor harboring GNAQ or GNA11 mutations. In another embodiment, the uveal melanoma is metastatic uveal melanoma harboring GNAQ or GNA11 mutations.

In another embodiment, Compound 1, or a pharmaceutically acceptable salt thereof, is administered at a dose of about 100 mg once daily (QD) of free base equivalent of Compound 1. In another embodiment, Compound 1, or a pharmaceutically acceptable salt thereof, is administered at a dose of about 200 mg once daily (QD) of free base equivalent of Compound 1. In yet another embodiment, Compound 1, or a pharmaceutically acceptable salt thereof, is administered at a dose of about 300 mg once daily (QD) of free base equivalent of Compound 1. In yet another embodiment, Compound 1, or a pharmaceutically acceptable salt thereof, is administered at a dose of about 400 mg once daily (QD) of free base equivalent of Compound 1.

In another embodiment, the administration of Compound 1, or a pharmaceutically acceptable salt thereof, is uninterrupted during a dosing schedule comprising at least one 7-day dosing cycle. In another embodiment, the administration of Compound 1, or a pharmaceutically acceptable salt thereof, is uninterrupted during a dosing schedule comprising at least four 7-day dosing cycles. In another embodiment, the administration of Compound 1, or a pharmaceutically acceptable salt thereof, is uninterrupted during a dosing schedule comprising at least eight 7-day dosing cycles. In another embodiment, the administration of Compound 1, or a pharmaceutically acceptable salt thereof, is uninterrupted during a dosing schedule comprising at least twelve, sixteen, or twenty 7-day dosing cycles. In another embodiment, the administration of Compound 1, or a pharmaceutically acceptable salt thereof, is uninterrupted during a dosing schedule comprising at least twenty-four 7-day dosing cycles.

In an embodiment, administering Compound 1, or a pharmaceutically acceptable salt thereof, reduces the size of one or more lesions of the uveal in said subject.

In another embodiment, administering Compound 1, or a pharmaceutically acceptable salt thereof, decreases the growth rate of one of more lesions of the uveal melanoma in said subject.

Exemplary lengths of time associated with the course of the treatment methods is about five years, about 4 years, about 3 years, about 2 years, about 1 year, about 11 months, about 10 months, about 9 months, about 8 months, about 7 months, about 6 months, about 5 months, about 4 months, about 3 months, about 2 months, or about 1 month.

Exemplary lengths of time associated with the course of the treatment methods is about five years and so on; or any days, weeks, months, or years in between; for example, a treatment cycle can include 5 months and additional weeks and/or days, or one year and additional months, weeks, and/or days, and the like.

In some embodiments, Compound 1, or a pharmaceutically acceptable salt thereof, is administered continuously (i.e., a continuous treatment until termination).

The UM to be treated can include one or more of a number of mutations, including a substitution mutation, an insertion mutation, and/or a deletion in GNAQ or GNA11 mutation. In some aspects, the GNAQ or GNA11 mutation is a gain of function mutation. In some aspects, the GNAQ or GNA11 mutation activates the PKC signaling pathway. In various aspects, the GNAQ or GNA11 mutation can be the substitution of glutamine in codon 209 (Q209) and/or a substitution of arginine in codon 183 (R183). The GNAQ or GNA11 mutation can be a substitution other than glutamine in codon 209 (Q209), other than a substitution of arginine in codon 183 (R183), or other than both. In some aspects, the GNAQ mutation is one of Q209P, Q209L, Q209H, Q209K, or Q209Y, or the GNA11 mutation is one of Q209P, Q209L, Q209K or Q209H. In further aspects, the GNAQ mutation can be R183Q, or the GNA11 mutation can be R183C or R183H. In yet further examples, the GNAQ or GNA11 mutation is at one or more of R256, L279, R166, A168, R210, R213, R166, A231, A342, D333, G171, R147, R73, T47, E191, E221, R149, T175, T379, T85, A86, E163, D195, E319, E191, E280, E49, P293, R300, R338, R60, D155, D205, D321, 1226, R37, or V240. In further examples, the UM can comprise one or more of a Q209P, Q209L, Q209H, Q209K, Q209Y, or R183Q mutation in GNAQ, or the UM can comprise one or more of a Q209P, Q209L, Q209H, or Q209K mutation in GNA11. Additional examples of mutations in GNAQ or GNA11 are described in WO 2020/146355, which is incorporated by reference herewith in its entirety.

Exemplary lengths of time associated with the course of the treatment methods disclosed herein include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about 4 months; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years and so on; or any days, weeks, months, or years in between; for example a treatment cycle can include 5 months and additional weeks and/or days, or one year and additional months, weeks, and/or days, and the like.

In an embodiment of the methods, the method involves the administration of a therapeutically effective amount of a compound provided herein, or a pharmaceutically acceptable salt thereof, to a subject (including, but not limited to a human or animal) in need of treatment (including a subject identified as in need).

In one embodiment of the foregoing methods, the treatment reduces the size of one of more lesions of the uveal melanoma.

In one embodiment of the foregoing methods, the treatment reduces the size of one of more lesions of the metastatic uveal melanoma in said subject.

In one embodiment of the foregoing methods, the treatment decreases the growth rate of one of more lesions of the uveal melanoma in said subject.

In one embodiment of the foregoing methods, the treatment decreases the growth rate of one of more lesions of the metastatic uveal melanoma in said subject.

In another embodiment, the subject is a First-Line MUM subject.

### Administration/Dosage/Formulations

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating uveal melanoma, comprising Compound 1, or an equivalent dose of a pharmaceutically acceptable salt thereof, in a unit dosage of about 400 mg to about 600 mg. In another embodiment, the present disclosure provides a kit for treating uveal melanoma, comprising Compound 1, or an equivalent dose of a pharmaceutically acceptable salt thereof, in an amount of about 300 mg. In another embodiment, the present disclosure provides a kit for treating uveal melanoma, comprising Compound 1, or an equivalent dose of a pharmaceutically acceptable salt thereof, in an amount of about 200 mg. In another embodiment, the present disclosure provides a kit for treating uveal melanoma, comprising Compound 1, or an equivalent dose of a pharmaceutically acceptable salt thereof, in an amount from about 400 mg per day to about 600 mg per day. In some embodiments, the kit further comprises packaging and instructions. In yet another embodiment, the uveal melanoma is metastatic uveal melanoma. In yet another embodiment, the metastatic uveal melanoma is a solid tumor harboring GNAQ or GNA11 mutations.

In certain embodiments, the kit comprises a pharmaceutical product comprising a pharmaceutical composition comprising Compound 1, or a pharmaceutically acceptable salt thereof, and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

The kits provided herein comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

### Non-Limiting Exemplary Embodiments

In further embodiments 1 to 99 below, the present disclosure includes:

1. In Embodiment 1 is a method of treating uveal melanoma in a patient having an ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure.

2. In Embodiment 2 is the method of embodiment 1, wherein the ocular tumor is defined by a set of dimensions comprising (i) greater than or equal to 5 mm largest basal diameter (LBD) and any apical height, or (ii) any LBD and greater than 3 mm apical height.

3. In embodiment 3 is a method of treating a patient having uveal melanoma with an ocular tumor with a size indicated for an interventional procedure comprising enucleation of the eye, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

4. In Embodiment 4 is a method of treating a patient having uveal melanoma with an ocular tumor with a size indicated for an interventional procedure comprising irradiating the ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

5. In Embodiment 5 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor to the patient as a neoadjuvant therapy;
b) reducing the size of the ocular tumor to dimensions of less than 5 mm LBD and less than 3 mm apical height; and
c) treating the reduced-size ocular tumor with an interventional procedure.

6. In Embodiment 6 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor to the patient; and
b) reducing the size of the ocular tumor defined by a set of dimensions having less than 5 mm LBD and less than 3 mm apical height.

7. In Embodiment 7, the method of embodiment 6, further comprising treating the reduced-size ocular tumor with an interventional procedure.

8. In Embodiment 8 is a method of treating uveal melanoma in a patient having an ocular tumor with a size defined by a set of dimensions comprising (i) greater than 16 mm LBD and any apical height, or (ii) any LBD and greater than 8 mm apical height, the method comprising
a) administering a PKC inhibitor to the patient as a neoadjuvant therapy;
b) reducing the size of the ocular tumor to dimensions of less than 16 mm LBD and less than 8 mm apical height; and
c) treating the reduced-size ocular tumor with an interventional procedure.

9. In Embodiment 9, the method of embodiment 8, wherein step (b) is reducing the size of the ocular tumor to dimensions of less than 16 mm LBD and 3-8 mm apical height.

10. In Embodiment 10 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor to the patient as a neoadjuvant therapy;
b) reducing the size of the ocular tumor defined by a set of dimensions having 5-19 mm LBD or less than 2.5 mm apical height; and
c) treating the reduced-size ocular tumor with an interventional procedure.

11. In Embodiment 11, the method of embodiment 10, wherein step (b) is reducing the size of the ocular tumor to dimensions of 5-19 mm LBD and less than 2.5 mm apical height.

12. In Embodiment 12 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor to the patient as a neoadjuvant therapy; and
b) reducing the size of the ocular tumor defined by a set of dimensions having 5-19 mm LBD and less than 2.5 mm apical height.

13. In Embodiment 13 is a method of treating uveal melanoma in a patient having an ocular tumor with a size defined by a set of dimensions comprising (i) up to 22 mm in LBD and (ii) up to 15 mm in apical height, the method comprising
a) administering a PKC inhibitor to the patient as a neoadjuvant therapy; and
b) reducing the size of the ocular tumor by at least 10%.

14. In Embodiment 14 is a method of treating uveal melanoma in a patient having an ocular tumor with a size defined by a set of dimensions comprising (i) up to 22 mm in LBD or (ii) up to 15 mm in apical height, the method comprising
a) administering a PKC inhibitor to the patient as a neoadjuvant therapy; and
b) reducing the size of the ocular tumor by at least 10%.

15. In Embodiment 15, the method of embodiment 13 or 14, wherein the patient had been recommended enucleation before the neoadjuvant therapy was administered.

16. In Embodiment 16 is a method of treating uveal melanoma in a patient having an ocular tumor with a size defined by a set of dimensions comprising (i) at least 6 mm in LBD and (ii) at least 3 mm in apical height, the method comprising
a) administering a PKC inhibitor to the patient as a neoadjuvant therapy; and
b) reducing the size of the ocular tumor by at least 10%.

17. In Embodiment 17 is a method of treating uveal melanoma in a patient having an ocular tumor with a size defined by a set of dimensions comprising (i) at least 6 mm in LBD or (ii) at least 3 mm in apical height, the method comprising
a) administering a PKC inhibitor to the patient as a neoadjuvant therapy; and
b) reducing the size of the ocular tumor by at least 10%.

18. In Embodiment 18, the method of embodiment 17, wherein the patients having sub-foveal or greater than 180-degree optic nerve involved tumors were excluded.

19. In Embodiment 19, the method of any one of embodiments 16 to 18, wherein the patient had been recommended irradiating the tumor before the neoadjuvant therapy was administered.

20. In Embodiment 20, method of any one of embodiments 12 to 19, further comprising treating the reduced-size ocular tumor with an interventional procedure.

21. In Embodiment 21 is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
a) administering a PKC inhibitor to the patient as a neoadjuvant therapy;
b) reducing the size of the ocular tumor to dimensions of less than or equal to 19 mm LBD and 2.5-10 mm apical height; and
c) treating the reduced-size ocular tumor with an interventional procedure.

22. In Embodiment 22, the method of any one of embodiments 1 to 21, wherein the interventional procedure comprises local surgical resection of the tumor, irradiating the tumor, or any combination thereof.

23. In Embodiment 23, the method of any one of embodiments 1 to 22, the interventional procedure comprises local surgical resection of the tumor.

24. In Embodiment 24, the method of any one of embodiments 1 to 21, wherein the interventional procedure comprises irradiating the tumor.

25. In Embodiment 25, the method of any one of embodiments 1 to 21, wherein the interventional procedure is irradiating the tumor.

26. In Embodiment 26, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor by at least 10%; and
d) irradiating the reduced-size ocular tumor with a second therapeutically effective dose of radiation less than the first therapeutically effective dose of radiation.

27. In Embodiment 27, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor; and
d) irradiating the reduced-size ocular tumor with a second therapeutically effective dose of radiation at least 10% less than the first therapeutically effective dose of radiation.

28. In Embodiment 28, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy to reduce the size of the ocular tumor by at least 10%; and
c) irradiating the reduced-size ocular tumor with a second therapeutically effective dose of radiation less than the first therapeutically effective dose of radiation.

29. In Embodiment 29, the method of Embodiment 28 wherein the second therapeutically effective dose of radiation is less than the first therapeutically effective dose of radiation by at least 5%, 10%, 15%, 20%, 25%, 30%, 35%, 40%, 45%, 50%, 55%, 60%, 65&, 70%, 75%, 80%, 85%, or 90%.

30. In Embodiment 30, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy to reduce the size of the ocular tumor; and
c) irradiating the reduced-size ocular tumor with a second therapeutically effective dose of radiation at least 10% less than the first therapeutically effective dose of radiation.

31. In Embodiment 31, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy over a period of time sufficient to reduce the size of the ocular tumor by at least 10%; and
c) irradiating the reduced-size ocular tumor with a second therapeutically effective dose of radiation less than the first therapeutically effective dose of radiation.

32. In Embodiment 32, the method of Embodiment 31 wherein the second therapeutically effective dose of radiation is less than the first therapeutically effective dose of radiation by at least 5%, 10%, 15%, 20%, 25%, 30%, 35%, 40%, 45%, 50%, 55%, 60%, 65&, 70%, 75%, 80%, 85%, or 90%. 33. In Embodiment 33, is a method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy over a period of time sufficient to reduce the size of the ocular tumor; and
c) irradiating the reduced-size ocular tumor with a second therapeutically effective dose of radiation at least 10% less than the first therapeutically effective dose of radiation.

34. In Embodiment 34, is a method of treating a patient having uveal melanoma with an ocular tumor of a size indicated for an interventional procedure comprising irradiating the ocular tumor, the method comprising:
a) administering a PKC inhibitor to the patient as neoadjuvant therapy;
b) reducing the size of the ocular tumor by at least 10%; and
c) irradiating the reduced-size ocular tumor with a therapeutically effective dose of radiation, wherein the therapeutically effective dose of radiation is less than a dose of radiation required to irradiate the ocular tumor in the absence of neoadjuvant therapy.

35. In Embodiment 35, the method of any one of embodiments 1 to 34, wherein the PKC inhibitor is Compound 1:

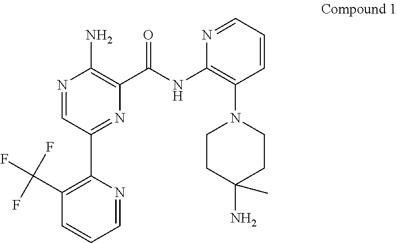 [SMILES: CC1(N)CCN(c2cccnc2NC(=O)c2nc(-c3ncccc3C(F)(F)F)cnc2N)CC1]

or a pharmaceutically acceptable salt thereof.

35A. In Embodiment 35A, the method of any one of embodiments 1 to 35, wherein the PKC inhibitor is Compound 1.

36. In Embodiment 36, the method of any one of embodiments 1 to 35, wherein the interventional procedure is a globe preserving treatment (GPT).

37. In Embodiment 37, the method of any one of embodiments 1 to 36, wherein the interventional procedure is selected from the group consisting of plaque brachytherapy (PBT), external beam radiotherapy (EBRT), stereotactic radiosurgery (SRS), LASER, additional pharmacological agent therapy, and any combination thereof.

38. In Embodiment 38, the method of any one of embodiments 1 to 36, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

39. In Embodiment 39, the method of any one of embodiments 1 to 36, wherein the interventional procedure is selected from the group consisting of proton beam radiotherapy, transpupillary thermotherapy (TTT), thermotherapy, laser photocoagulation therapy, limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), charged-particles beam radiotherapy, and any combination thereof.

40. In Embodiment 40, the method of any one of embodiments 1 to 36, wherein the interventional procedure is brachytherapy (BT).

41. In Embodiment 41, the method of any one of embodiments 38 to 40, wherein stereotactic radiotherapy (SRS) is selected from the group consisting of gamma-knife, cyber knife and linear accelerator (LINAC).

42. In Embodiment 42, the method of any one of embodiments 1 to 41, wherein the method further comprises continuing administering a PKC inhibitor to the patient as an adjuvant therapy during the period of the interventional procedure.

43. In Embodiment 43, the method of any one of embodiments 1 to 41, wherein the method further comprises continuing administering a PKC inhibitor to the patient as an adjuvant therapy after the completion of the interventional procedure.

44. In Embodiment 44, the method of embodiment 43, wherein the adjuvant therapy is administered at least 1 week, at least 2 weeks, at least 3 weeks, at least 4 weeks, at least 5 weeks, or at least 6 weeks after the completion of the interventional procedure.

45. In Embodiment 45, the method of embodiment 43 or 44, wherein the adjuvant therapy is administered at least 4 weeks, at least 5 weeks, or at least 6 weeks after the completion of the interventional procedure.

46. In Embodiment 46, the method of any one of embodiments 1 to 45, wherein the PKC inhibitor is administered orally.

47. In Embodiment 47, the method of any one of embodiments 1 to 46, wherein method further comprises preserving the eye globe which had the ocular tumor.

48. In Embodiment 48, the method of any one of embodiments 1 to 47, wherein the method further comprises preserving a function of an eye which had the ocular tumor.

49. In Embodiment 49, the method of embodiment 48, wherein preserving the function of the eye which had the ocular tumor comprises at least partially preserving the eye function.

50. In Embodiment 50, the method of any one of embodiments 1 to 49, wherein method further comprises preventing irreversible vision loss.

51. In Embodiment 51, the method of any one of embodiments 1 to 49, wherein method further comprises preserving visual symptoms.

52. In Embodiment 52, the method of any one of embodiments 1 to 49, wherein method further comprises preserving depth perception.

53. In Embodiment 53, the method of any one of embodiments 1 to 52, wherein method further comprises preserving vision for greater than at least 1 year.

54. In Embodiment 54, the method of any one of embodiments 1 to 53, wherein method further comprises preventing metastasis.

55. In Embodiment 55, the method of any one of embodiments 1 to 53, wherein method further comprises delaying onset of metastasis.

56. In Embodiment 56, the method of any one of embodiments 1 to 55, wherein the ocular tumor is spatially separated from the optic nerve.

57. In Embodiment 57, the method of any one of embodiments 1 to 56, wherein the ocular tumor, prior to administering the neoadjuvant therapy, has a size from 5 mm to 19 mm LBD and less than 2.5 mm apical height.

58. In Embodiment 58, the method of any one of embodiments 1 to 56, wherein the ocular tumor, prior to administering the neoadjuvant therapy, has a size less than or equal to 19 mm LBD and 2.5-10 mm apical height.

59. In Embodiment 59, the method of any one of embodiments 1 to 56, wherein the ocular tumor, prior to administering the neoadjuvant therapy, has a size greater than 19 mm LBD.

60. In Embodiment 60, the method of any one of embodiments 1 to 56, wherein the ocular tumor, prior to administering the neoadjuvant therapy, has a size greater than 10 mm apical height and any size LBD.

61. In Embodiment 61, the method of any one of embodiments 1 to 56, wherein the ocular tumor, prior to administering the neoadjuvant therapy, has a size greater than 8 mm apical height and the tumor is located close to the optic nerve.

62. In Embodiment 62, the method of any one of embodiments 1 to 56, wherein the ocular tumor, prior to administering the neoadjuvant therapy, has a size of greater than 6 mm and less than 12 mm apical height, and not greater than 16 mm LBD.

63. In Embodiment 63, the method of any one of embodiments 1 to 56, wherein the ocular tumor, prior to administering the neoadjuvant therapy, has a size of greater than 3 mm and less than 8 mm apical height, and not greater than 16 mm LBD.

64. In Embodiment 64, the method of any one of embodiments 1 to 56, wherein the ocular tumor, prior to administering the neoadjuvant therapy, has a size of greater than 8 mm apical height, and greater than 16 mm LBD.

65. In Embodiment 65, the method of any one of embodiments 1 to 64, wherein the ocular tumor, prior to administering the neoadjuvant therapy, is not greater than 16 mm LBD.

66. In Embodiment 66, the method of any one of embodiments 1 to 65, wherein the size of the ocular tumor, prior to administering the neoadjuvant therapy, is greater than 10 mm in apical height.

67. In Embodiment 67, the method of any one of embodiments 1 to 66, wherein the size of the ocular tumor after administering the neoadjuvant therapy but prior to the interventional procedure is reduced by at least 20%.

68. In Embodiment 68, the method of any one of embodiments 1 to 67, wherein the size of the ocular tumor after administering the neoadjuvant therapy but prior to the interventional procedure is reduced by at least 30%.

69. In Embodiment 69, the method of any one of embodiments 1 to 68, wherein the size of the ocular tumor after administering the neoadjuvant therapy but prior to the interventional procedure is reduced by at least 50%.

70. In Embodiment 70, the method of any one of embodiments 1 to 69, wherein the PKC inhibitor, or an equivalent dose of a pharmaceutically acceptable salt thereof, is administered at a dose of about 100 to about 400 mg two times per day (BID).

71. In Embodiment 71, the method of any one of embodiments 1 to 70, wherein the PKC inhibitor, or an equivalent dose of a pharmaceutically acceptable salt thereof, is administered at a dose of about 100 mg BID, about 150 mg BID, about 200 mg BID, about 250 mg BID, about 300 mg BID, about 350 mg BID, or about 400 mg two times per day (BID).

72. In Embodiment 72, the method of any one of embodiments 1 to 71, wherein the PKC inhibitor, or an equivalent dose of a pharmaceutically acceptable salt thereof, is administered at a dose of about 200 to about 400 mg two times per day (BID).

73. In Embodiment 73, the method of any one of embodiments 1 to 72, wherein the PKC inhibitor, or an equivalent dose of a pharmaceutically acceptable salt thereof, is administered at a dose of about 300 mg two times per day (BID).

74. In Embodiment 74, the method of any one of embodiments 1 to 72, wherein the PKC inhibitor, or an equivalent dose of a pharmaceutically acceptable salt thereof, is administered at a dose of 200 mg two times per day (BID).

75. In Embodiment 75, the method of any one of embodiments 1 to 72, wherein the PKC inhibitor, or an equivalent dose of a pharmaceutically acceptable salt thereof, is administered at a dose of about 400 mg to about 600 mg daily.

76. In Embodiment 76, the method of any one of embodiments 1 to 69, wherein the PKC inhibitor, or an equivalent dose of a pharmaceutically acceptable salt thereof, is administered at a dose of about 200 mg to about 600 mg daily.

77. In Embodiment 77, the method of any one of embodiments 1 to 76, wherein the PKC inhibitor, or an equivalent dose of a pharmaceutically acceptable salt thereof is administered daily for at least 7 days, at least 14 days, at least 21, days, at least 28 days, at least 56 days, at least 84 days, at least, 112 days, at last 140 days, or at least 168 days.

78. In Embodiment 78, the method of any one of embodiments 1 to 77, wherein the PKC inhibitor, or an equivalent dose of a pharmaceutically acceptable salt thereof is administered daily for at least 7 days, at least 14 days, at least 21, days, or at least 28 days.

79. In Embodiment 79, the method of any one of embodiments 1 to 77, wherein the PKC inhibitor, or an equivalent dose of a pharmaceutically acceptable salt thereof is administered daily for at least 28 days, at least 56 days, at least 84 days, at least 140 days, or at least 168 days.

80. In Embodiment 80, the method of any one of embodiments 1 to 76, wherein the PKC inhibitor, or an equivalent dose of a pharmaceutically acceptable salt thereof is administered daily for at least 1 month, at least 2 months, at least 3 months, at least 4 months, at least 5 months or at least 6 months.

81. In Embodiment 81, the method of any one of embodiments 1 to 80, wherein the interventional procedure is selected from the group consisting of plaque brachytherapy (PBT), external beam radiotherapy (EBRT), stereotactic radiosurgery (SRS), and any combination thereof.

82. In Embodiment 82, the method of any one of embodiments 1 to 81, wherein the interventional procedure is plaque brachytherapy (PBT).

83. In Embodiment 83, the method of any one of embodiments 1 to 82, wherein the uveal melanoma is a solid tumor harboring GNAQ or GNA11 mutations.

84. In Embodiment 84, the method of any one of embodiments 1 to 83, wherein the patient has additional non-ocular tumor.

85. In Embodiment 85, the method of embodiment 84, wherein the non-ocular tumor is metastatic uveal melanoma.

86. In Embodiment 86, the method of any one of embodiments 1 to 85, wherein the ocular tumor is not malignant.

87. In Embodiment 87, the method of any one of embodiments 1 to 85, wherein the ocular tumor is malignant.

88. In Embodiment 88, the method of any one of embodiments 1 to 87, wherein the ocular tumor is intraocular tumor.

89. In Embodiment 89, the method of any one of embodiments 1 to 88, wherein the patient is human leukocyte antigen (HLA)-A2 negative.

90. In Embodiment 90, the method of any one of embodiments 1 to 88, wherein the patient is human leukocyte antigen (HLA)-A2 positive.

91. In Embodiment 91, the method of any one of embodiments 1 to 90, wherein the patient has not been previously treated with a PKC inhibitor.

92. In Embodiment 92, the method of any one of above embodiments, wherein the size of the ocular tumor is measured using RECIST 1.1 criteria.

93. In Embodiment 93, the method of any one of above embodiments, wherein the size of the ocular tumor is determined by CT scan, MRI, or ultrasound, or a combination thereof.

94. In Embodiment 94, the method of any one of above embodiments, wherein the size of the ocular tumor is determined by CT scan.

95. In Embodiment 95, the method of any one of above embodiments, wherein the size of the ocular tumor is determined by MRI.

96. In Embodiment 96, the method of any one of above embodiments, wherein the size of the ocular tumor is determined by ultrasound.

97. In Embodiment 97, the method of any one of above embodiments, wherein the neoadjuvant therapy is administered to the patient for at least 1 month, at least 2 months, at least 3 months, at least 4 months, at least 5 months, or at least 6 months.

99. In Embodiment 99, the method of any one of above embodiments, wherein the adjuvant therapy is administered to the patient for at least 1 month, at least 2 months, at least 3 months, at least 4 months, at least 5 months, or at least 6 months.

### Non-Limiting Exemplary Embodiments

1. In Embodiment 1 is a method of treating uveal melanoma in a patient having an ocular tumor, the method comprising
a) treating the ocular tumor with an interventional procedure, and
b) administering a PKC inhibitor to the patient as an adjuvant therapy.

2. In Embodiment 2 is the method of embodiment 1, wherein the ocular tumor is defined by a set of dimensions comprising (i) greater than or equal to 5 mm largest basal diameter (LBD) and any apical height, or (ii) any LBD and greater than 3 mm apical height.

3. In embodiment 3 is a method of treating a patient having uveal melanoma with an ocular tumor with a size indicated for an interventional procedure comprising enucleation of the eye, the method comprising
a) treating the ocular tumor with an interventional procedure other than enucleation, and
b) administering a PKC inhibitor to the patient as an adjuvant therapy.

4. In Embodiment 4 is a method of treating a patient having uveal melanoma with an ocular tumor with a size indicated for an interventional procedure comprising irradiating the ocular tumor, the method comprising
a) treating the ocular tumor with an interventional procedure other than enucleation, and
b) administering a PKC inhibitor to the patient as an adjuvant therapy.

5. In Embodiment 5 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) treating the ocular tumor with an interventional procedure, and
b) administering a PKC inhibitor to the patient as an adjuvant therapy.

6. In Embodiment 6 is a method of treating uveal melanoma in a patient having an ocular tumor with a size defined by a set of dimensions comprising (i) greater than 16 mm LBD and any apical height, or (ii) any LBD and greater than 8 mm apical height, the method comprising
a) treating the ocular tumor with an interventional procedure, and
b) administering a PKC inhibitor to the patient as an adjuvant therapy.

7. In Embodiment 7 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) treating the ocular tumor with an interventional procedure, and
b) administering a PKC inhibitor to the patient as an adjuvant therapy.

8. In Embodiment 8 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) treating the ocular tumor with an interventional procedure, and
b) administering a PKC inhibitor to the patient as an adjuvant therapy.

9. In Embodiment 9 is a method of treating uveal melanoma in a patient having an ocular tumor with a size defined by a set of dimensions comprising (i) up to 22 mm in LBD and (ii) up to 15 mm in apical height, the method comprising
a) treating the ocular tumor with an interventional procedure, and
b) administering a PKC inhibitor to the patient as an adjuvant therapy.

10. In Embodiment 10 is a method of treating uveal melanoma in a patient having an ocular tumor with a size defined by a set of dimensions comprising (i) up to 22 mm in LBD or (ii) up to 15 mm in apical height, the method comprising
a) treating the ocular tumor with an interventional procedure, and
b) administering a PKC inhibitor to the patient as an adjuvant therapy.

11. In Embodiment 11 is a method of treating uveal melanoma in a patient having an ocular tumor with a size defined by a set of dimensions comprising (i) at least 6 mm in LBD and (ii) at least 3 mm in apical height, the method comprising
a) treating the ocular tumor with an interventional procedure, and
b) administering a PKC inhibitor to the patient as an adjuvant therapy.

12. In Embodiment 12 is a method of treating uveal melanoma in a patient having an ocular tumor with a size defined by a set of dimensions comprising (i) at least 6 mm in LBD or (ii) at least 3 mm in apical height, the method comprising
a) treating the ocular tumor with an interventional procedure, and
b) administering a PKC inhibitor to the patient as an adjuvant therapy.

13. In Embodiment 13, the method of Embodiment 12, wherein the patients having sub-foveal or greater than 180-degree optic nerve involved tumors were excluded.

14. In Embodiment 14 is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
a) treating the ocular tumor with an interventional procedure, and
b) administering a PKC inhibitor to the patient as an adjuvant therapy.

15. In Embodiment 15, the method of any one of embodiments 1 to 14, wherein the interventional procedure comprises local surgical resection of the tumor, irradiating the tumor, or any combination thereof.

16. In Embodiment 16, the method of any one of embodiments 1 to 14, the interventional procedure comprises local surgical resection of the tumor.

17. In Embodiment 17, the method of any one of embodiments 1 to 14, wherein the interventional procedure comprises irradiating the tumor.

18. In Embodiment 18, the method of any one of embodiments 1 to 14, wherein the interventional procedure is irradiating the tumor.

19. In Embodiment 19, the method of any one of embodiments 1 to 18, wherein the PKC inhibitor is Compound 1:

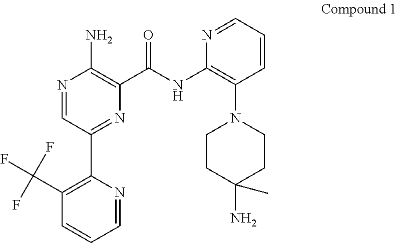 [SMILES: CC1(N)CCN(c2cccnc2NC(=O)c2nc(-c3ncccc3C(F)(F)F)cnc2N)CC1]

or a pharmaceutically acceptable salt thereof.

20. In Embodiment 20, the method of any one of embodiments 1 to 19, wherein the interventional procedure is a globe preserving treatment (GPT).

21. In Embodiment 21, the method of any one of embodiments 1 to 20, wherein the interventional procedure is selected from the group consisting of plaque brachytherapy (PBT), external beam radiotherapy (EBRT), stereotactic radiosurgery (SRS), LASER, additional pharmacological agent therapy, and any combination thereof.

22. In Embodiment 22, the method of any one of embodiments 1 to 20, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

23. In Embodiment 23, the method of any one of embodiments 1 to 20, wherein the interventional procedure is selected from the group consisting of proton beam radiotherapy, transpupillary thermotherapy (TTT), thermotherapy, laser photocoagulation therapy, limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), charged-particles beam radiotherapy, and any combination thereof.

24. In Embodiment 24, the method of any one of embodiments 1 to 23, wherein the interventional procedure is brachytherapy (BT).

25. In Embodiment 25, the method of any one of embodiments 22 to 24, wherein stereotactic radiotherapy (SRS) is selected from the group consisting of gamma-knife, cyber knife, and linear accelerator (LINAC).

26. In Embodiment 26, the method of any one of embodiments 1 to 25, wherein the method comprises administering a PKC inhibitor to the patient as an adjuvant therapy during the period of the interventional procedure.

27. In Embodiment 27, the method of any one of embodiments 1 to 25, wherein the method comprises administering a PKC inhibitor to the patient as an adjuvant therapy after the completion of the interventional procedure.

28. In Embodiment 28, the method of embodiment 27, wherein the adjuvant therapy is administered at least 1 week, at least, 2 weeks, at least 3 weeks, at least 4 weeks, at least 5 weeks, or at least 6 weeks after the completion of the interventional procedure.

29. In Embodiment 29, the method of embodiment 27 or 28, wherein the adjuvant therapy is administered at least 4 weeks, at least 5 weeks, or at least 6 weeks after the completion of the interventional procedure.

30. In Embodiment 30, the method of any one of embodiments 1 to 29, wherein the PKC inhibitor is administered orally.

31. In Embodiment 31, the method of any one of embodiments 1 to 30, wherein method further comprises preserving the eye globe which had the ocular tumor.

32. In Embodiment 32, the method of any one of embodiments 1 to 31, wherein the method further comprises preserving a function of an eye which had the ocular tumor.

33. In Embodiment 33, the method of embodiment 32, wherein preserving the function of the eye which had the ocular tumor comprises at least partially preserving the eye function.

34. In Embodiment 34, the method of any one of embodiments 1 to 33, wherein method further comprises preventing irreversible vision loss.

35. In Embodiment 35, the method of any one of embodiments 1 to 34, wherein method further comprises preserving visual symptoms.

36. In Embodiment 36, the method of any one of embodiments 1 to 35, wherein method further comprises preserving depth perception.

37. In Embodiment 37, the method of any one of embodiments 1 to 36, wherein method further comprises preserving vision for greater than at least 1 year.

38. In Embodiment 38, the method of any one of embodiments 1 to 37, wherein method further comprises preventing metastasis.

39. In Embodiment 39, the method of any one of embodiments 1 to 37, wherein method further comprises delaying onset of metastasis.

40. In Embodiment 40, the method of any one of embodiments 1 to 39, wherein the ocular tumor is spatially separated from the optic nerve.

41. In Embodiment 41, the method of any one of embodiments 1 to 39, wherein the ocular tumor, prior to the interventional procedure, has a size from 5 mm to 19 mm LBD and less than 2.5 mm apical height.

42. In Embodiment 42, the method of any one of embodiments 1 to 39, wherein the ocular tumor, prior to the interventional procedure, has a size less than or equal to 19 mm LBD and 2.5-10 mm apical height.

43. In Embodiment 43, the method of any one of embodiments 1 to 39, wherein the ocular tumor, prior to the interventional procedure, has a size greater than 19 mm LBD.

44. In Embodiment 44, the method of any one of embodiments 1 to 39, wherein the ocular tumor, prior to the interventional procedure, has a size greater than 10 mm apical height and any size LBD.

45. In Embodiment 45, the method of any one of embodiments 1 to 39, wherein the ocular tumor, prior to the interventional procedure, has a size greater than 8 mm apical height and the tumor is located close to the optic nerve.

46. In Embodiment 46, the method of any one of embodiments 1 to 39, wherein the ocular tumor, prior to the interventional procedure, has a size of greater than 6 mm and less than 12 mm apical height, and not greater than 16 mm LBD.

47. In Embodiment 47, the method of any one of embodiments 1 to 39, wherein the ocular tumor, prior to the interventional procedure, has a size of greater than 3 mm and less than 8 mm apical height, and not greater than 16 mm LBD.

48. In Embodiment 48, the method of any one of embodiments 1 to 39, wherein the ocular tumor, prior to the interventional procedure, has a size of greater than 8 mm apical height, and greater than 16 mm LBD.

49. In Embodiment 49, the method of any one of embodiments 1 to 39, wherein the ocular tumor, prior to the interventional procedure, is not greater than 16 mm LBD.

50. In Embodiment 50, the method of any one of embodiments 1 to 39, wherein the size of the ocular tumor, prior to the interventional procedure, is greater than 10 mm in apical height.

51. In Embodiment 51, the method of any one of embodiments 1 to 50, wherein the size of the ocular tumor after the interventional procedure is reduced by at least 10%.

52. In Embodiment 52, the method of any one of embodiments 1 to 51, wherein the size of the ocular tumor after the interventional procedure is reduced by at least 20%.

53. In Embodiment 53, the method of any one of embodiments 1 to 52, wherein the size of the ocular tumor after the interventional procedure is reduced by at least 30%.

54. In Embodiment 54, the method of any one of embodiments 1 to 53, wherein the size of the ocular tumor after the interventional procedure is reduced by at least 50%.

55. In Embodiment 55, the method of any one of embodiments 1 to 54, wherein the PKC inhibitor, or an equivalent dose of a pharmaceutically acceptable salt thereof, is administered at a dose of about 100 to about 400 mg two times per day (BID).

56. In Embodiment 56, the method of any one of embodiments 1 to 55, wherein the PKC inhibitor, or an equivalent dose of a pharmaceutically acceptable salt thereof, is administered at a dose of about 100 mg BID, about 150 mg BID, about 200 mg BID, about 250 mg BID, about 300 mg BID, about 350 mg BID, or about 400 mg two times per day (BID).

57. In Embodiment 57, the method of any one of embodiments 1 to 56, wherein the PKC inhibitor, or an equivalent dose of a pharmaceutically acceptable salt thereof, is administered at a dose of about 200 to about 400 mg two times per day (BID).

58. In Embodiment 58, the method of any one of embodiments 1 to 57, wherein the PKC inhibitor, or an equivalent dose of a pharmaceutically acceptable salt thereof, is administered at a dose of about 300 mg two times per day (BID).

59. In Embodiment 59, the method of any one of embodiments 1 to 54, wherein the PKC inhibitor, or an equivalent dose of a pharmaceutically acceptable salt thereof, is administered at a dose of about 400 mg to about 600 mg daily.

60. In Embodiment 60, the method of any one of embodiments 1 to 54, wherein the PKC inhibitor, or an equivalent dose of a pharmaceutically acceptable salt thereof, is administered at a dose of about 200 mg to about 600 mg daily.

61. In Embodiment 61, the method of any one of embodiments 1 to 60, wherein the PKC inhibitor, or an equivalent dose of a pharmaceutically acceptable salt thereof is administered daily for at least 7 days, at least 14 days, at least 21, days, at least 28 days, at least 56 days, at least 84 days, at least, 112 days, at last 140 days, or at least 168 days.

62. In Embodiment 62, the method of any one of embodiments 1 to 61, wherein the PKC inhibitor, or an equivalent dose of a pharmaceutically acceptable salt thereof is administered daily for at least 7 days, at least 14 days, at least 21, days, or at least 28 days.

63. In Embodiment 63, the method of any one of embodiments 1 to 61, wherein the PKC inhibitor, or an equivalent dose of a pharmaceutically acceptable salt thereof is administered daily for at least 28 days, at least 56 days, at least 84 days, at least 140 days, or at least 168 days.

64. In Embodiment 64, the method of any one of embodiments 1 to 60, wherein the PKC inhibitor, or an equivalent dose of a pharmaceutically acceptable salt thereof is administered daily for at least 1 month, at least 2 months, at least 3 months, at least 4 months, at least 5 months or at least 6 months.

65. In Embodiment 65, the method of any one of embodiments 1 to 64, wherein the interventional procedure is selected from the group consisting of plaque brachytherapy (PBT), external beam radiotherapy (EBRT), stereotactic radiosurgery (SRS), and any combination thereof.

66. In Embodiment 66, the method of any one of embodiments 1 to 65, wherein the interventional procedure is plaque brachytherapy (PBT).

67. In Embodiment 67, the method of any one of embodiments 1 to 66, wherein the uveal melanoma is a solid tumor harboring GNAQ or GNA11 mutations.

68. In Embodiment 68, the method of any one of embodiments 1 to 67, wherein the patient has additional non-ocular tumor.

69. In Embodiment 69, the method of embodiment 68, wherein the non-ocular tumor is metastatic uveal melanoma.

70. In Embodiment 70, the method of any one of embodiments 1 to 69, wherein the ocular tumor is not malignant.

71. In Embodiment 71, the method of any one of embodiments 1 to 69, wherein the ocular tumor is malignant.

72. In Embodiment 72, the method of any one of embodiments 1 to 71, wherein the ocular tumor is intraocular tumor.

73. In Embodiment 73, the method of any one of embodiments 1 to 72, wherein the patient is human leukocyte antigen (HLA)-A2 negative.

74. In Embodiment 74, the method of any one of embodiments 1 to 72, wherein the patient is human leukocyte antigen (HLA)-A2 positive.

75. In Embodiment 75, the method of any of embodiments 1 to 74, wherein the patient has not been previously treated with a PKC inhibitor.

76. In Embodiment 76, the method of any of above embodiments, wherein the size of the ocular tumor is measured using RECIST 1.1 criteria.

77. In Embodiment 77, the method of any of above embodiments, wherein the size of the ocular tumor is determined by CT scan, MRI, or ultrasound, or a combination thereof.

78. In Embodiment 78, the method of any of above embodiments, wherein the size of the ocular tumor is determined by CT scan.

79. In Embodiment 79, the method of any of above embodiments, wherein the size of the ocular tumor is determined by MRI.

80. In Embodiment 80, the method of any of above embodiments, wherein the size of the ocular tumor is determined by ultrasound.

### Non-Limiting Embodiments

1. A method of treating uveal melanoma in a patient having an ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure.

2. The method of embodiment 1, wherein the ocular tumor is defined by a set of dimensions comprising (i) greater than or equal to 5 mm largest basal basal diameter (LBD) and any apical height, or (ii) any LBD and greater than 3 mm apical height.

3. A method of treating a patient having uveal melanoma with an ocular tumor with a size indicated for an interventional procedure comprising enucleation of the eye, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

4. The method of embodiment 3, wherein the ocular tumor is defined by a set of dimensions comprising (i) up to 22 mm in LBD and/or (ii) up to 15 mm in apical height.

5. A method of treating a patient having uveal melanoma with an ocular tumor with a size indicated for an interventional procedure comprising irradiating the ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

6. The method of embodiment 5, wherein the ocular tumor is defined by a set of dimensions comprising (i) at least 6 mm in LBD and/or (ii) at least 3 mm in apical height.

7. The method of any one of embodiments 1 to 6, wherein the interventional procedure comprises local surgical resection of the tumor, irradiating the tumor, or any combination thereof.

8. The method of any one of embodiments 1 to 7, wherein the interventional procedure comprises irradiating the tumor.

9. A method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor; and
d) irradiating the reduced-size ocular tumor with a second therapeutically effective dose of radiation less than the first therapeutically effective dose of radiation.

10. The method of embodiment 9, wherein in step (c) the ocular tumor is reduced by at least 10%.

11. The method of embodiment 9 or 10, wherein the ocular tumor before step (a) is defined by a set of dimensions comprising (i) at least 6 mm in LBD and/or (ii) at least 3 mm in apical height.

12. The method of any one of embodiments 9 to 11, wherein in step (d) the second therapeutically effective dose of radiation is at least 10% less than the first therapeutically effective dose of radiation.

13. The method of any one of embodiments 1 to 12, wherein the PKC inhibitor is Compound 1:

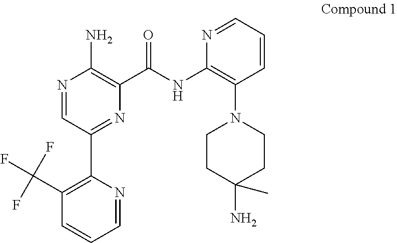 [SMILES: CC1(N)CCN(c2cccnc2NC(=O)c2nc(-c3ncccc3C(F)(F)F)cnc2N)CC1]

or a pharmaceutically acceptable salt thereof.

14. The method of any one of embodiments 1 to 13, wherein the interventional procedure is a globe preserving treatment (GPT).

15. The method of any one of embodiments 1 to 14, wherein the interventional procedure is selected from the group consisting of plaque brachytherapy (PBT), external beam radiotherapy (EBRT), stereotactic radiosurgery (SRS), and any combination thereof.

16. The method of any one of embodiments 1 to 15, wherein the interventional procedure is plaque brachytherapy (PBT).

17. The method of any one of embodiments 1 to 14, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

18. The method of any one of embodiments 1 to 16, wherein the method further comprises continuing administering a PKC inhibitor to the patient as an adjuvant therapy during the period of the interventional procedure.

19. The method of any one of embodiments 1 to 16, wherein the method further comprises continuing administering a PKC inhibitor as an adjuvant therapy after the completion of the interventional procedure.

20. The method of embodiment 19, wherein the adjuvant therapy is administered at least 4 weeks, at least 5 weeks, or at least 6 weeks after the completion of the interventional procedure.

21. The method of any one of embodiments 1 to 20, wherein the PKC inhibitor is administered orally.

22. The method of any one of embodiments 1 to 21, wherein the method further comprises preserving the eye globe which had the ocular tumor.

23. The method of any one of embodiments 1 to 22, wherein the method further comprises preserving a function of the eye which had the ocular tumor.

24. The method of embodiment 23, wherein preserving the function of the eye comprises at least partially preserving the eye function.

25. The method of any one of embodiments 1 to 24, wherein the method further comprises preventing irreversible vision loss.

26. The method of any one of embodiments 1 to 24, wherein the method further comprises preserving visual symptoms.

27. The method of any one of embodiments 1 to 24, wherein the method further comprises preserving depth perception.

28. The method of any one of embodiments 1 to 27, wherein the method further comprises preserving vision for greater than at least 1 year.

29. The method of any one of embodiments 1 to 28, wherein the method further comprises preventing metastasis.

30. The method of any one of embodiments 1 to 28, wherein the method further comprises delaying onset of metastasis.

31. The method of any one of embodiments 1 to 30, wherein the ocular tumor is spatially separated from the optic nerve.

32. The method of any one of embodiments 1 to 31, wherein the ocular tumor, prior to administering the neoadjuvant therapy, has a size
i) from 5 mm to 19 mm LBD and less than 2.5 mm apical height,
ii) less than or equal to 19 mm LBD and 2.5-10 mm apical height,
iii) greater than 19 mm LBD,
iv) greater than 10 mm apical height and any size LBD,
v) greater than 8 mm apical height and the tumor is located close to the optic nerve,
vi) greater than 6 mm and less than 12 mm apical height, and not greater than 16 mm LBD,
vii) greater than 3 mm and less than 8 mm apical height, and not greater than 16 mm LBD,
viii) greater than 8 mm apical height, and greater than 16 mm LBD,
ix) not greater than 16 mm LBD, or
x) greater than 10 mm in apical height.

33. The method of any one of embodiments 1 to 32, wherein the size of the ocular tumor after administering the neoadjuvant therapy but prior to the interventional procedure is reduced by at least 20%.

34. The method of any one of embodiments 1 to 33, wherein the PKC inhibitor, or an equivalent dose of a pharmaceutically acceptable salt thereof, is administered at a dose of about 200 mg to about 400 mg two times per day (BID).

35. The method of any one of embodiments 1 to 34, wherein the PKC inhibitor, or an equivalent dose of a pharmaceutically acceptable salt thereof, is administered at a dose of about 300 mg two times per day (BID).

36. The method of any one of embodiments 1 to 35, wherein the PKC inhibitor, or an equivalent dose of a pharmaceutically acceptable salt thereof, is administered at a dose of about 400 mg to about 600 mg daily.

37. The method of any one of embodiments 1 to 36, wherein the PKC inhibitor, or an equivalent dose of a pharmaceutically acceptable salt thereof is administered daily for at least 7 days, at least 14 days, at least 21, days, at least 28 days, at least 56 days, at least 84 days, at least, 112 days, at last 140 days, or at least 168 days.

38. The method of any one of embodiments 1 to 37, wherein the uveal melanoma is a solid tumor harboring GNAQ or GNA11 mutations.

39. The method of any one of embodiments 1 to 38, wherein the ocular tumor is intraocular tumor.

40. The method of any one of embodiments 1 to 39, wherein the size of the ocular tumor is measured using RECIST 1.1 criteria.

41. The method of any one of embodiments 1 to 40, wherein the size of the ocular tumor is determined by CT scan, MRI, or ultrasound, or a combination thereof.

42. The method of any one of embodiments 1 to 36 and 38 to 41, wherein the neoadjuvant therapy is administered to the patient for at least 1 month, at least 2 months, at least 3 months, at least 4 months, at least 5 months, or at least 6 months.

43. The method of any one of embodiments 1 to 36 and 38 to 42, wherein the adjuvant therapy is administered to the patient for at least 1 month, at least 2 months, at least 3 months, at least 4 months, at least 5 months, or at least 6 months.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2016/020864, the content of which is incorporated in its entirety.

### Example 1. Patient Inclusion/Exclusion Criteria

### Inclusion Criteria

Patients can meet all of the following inclusion criteria:
Patient must be at least 18 years of age.
Patient is able to provide written, informed consent before initiation of any study related-procedures, and is able, in the opinion of the investigator, to comply with all the requirements of the study.
Primary diagnosis of uveal melanoma as clinically determined by the treating investigator planned to undergo enucleation (prior plaque brachytherapy is permitted)
Life expectancy >3 months.
Able to safely swallow orally administered medication.
Patients with a prior history of or clinically stable concurrent malignancy are eligible for enrolment provided the malignancy is clinically insignificant, no treatment is required, and the patient is clinically stable.
   Patients with a history of squamous or basal cell carcinoma of the skin or carcinoma in the situ of the cervix may be enrolled.
   Patients with prostate cancer with an elevated PSA not requiring treatment may be enrolled.
Patient has Eastern Cooperative Oncology Group (ECOG) performance status 0-1 (Karnofsky 70%).
Patient has adequate organ function at screening:
   Absolute neutrophil count ≥1500/mm3 without the use of hematopoietic growth factors
   Platelet count ≥75,000/mm3 (must be at least 2 weeks post-platelet transfusion and not receiving platelet-stimulating agents)
   Haemoglobin ≥8.0 g/dL (must be at least 2 weeks post-red blood cell transfusion and not receiving erythropoietic-stimulating agents)
   Total bilirubin ≤1.5× the upper limit of normal (ULN). For patients with documented Gilbert's disease, total bilirubin <3.0 mg/dL is allowed
   Alanine aminotransferase (ALT) and aspartate aminotransferase (AST)≤3×ULN in the absence of documented liver metastases
   Serum albumin ≥2.0 umol/L
   Creatinine Clearance ≥60 mL/min/1.73 m2 by Cockroft-Gault equation
   Prothrombin time/International Normalized Ratio (INR) or partial thromboplastin time test results at screening ≤1.5×ULN (this applies only to patients who do not receive therapeutic anticoagulation; patients receiving therapeutic anticoagulation should be on a stable dose for at least 4 weeks prior to the first dose of study drug).
Female patients of childbearing potential must be non-pregnant, nonlactating, and have a negative serum human chorionic gonadotropin pregnancy test result within 28 days prior to the first study drug administration
   Females of childbearing potential who are sexually active with a non-sterilized male partner agree to use effective methods of contraception from screening, throughout the study drug and agree to continue using such precautions for 30 days after the final dose of study drug.
   Non-sterilized males who are sexually active with a female of childbearing potential must agree to use effective methods of contraception from Day 1 throughout the study drug and for 30 days after the final dose of study drug.

### Exclusion Criteria

The presence of any of the following can exclude a patient from being eligible for the study:
Previous treatment with a PKC inhibitor
Have adverse events (AEs) from prior anti-cancer therapy that have not resolved to Grade ≤1 except for alopecia, prior peripheral neuropathy, or anemia. Endocrinopathies resulting from previous immunotherapy are considered part of the medical history and not an AE.
Untreated or symptomatic malignant lesions in the central nervous system (CNS).
Known human immunodeficiency virus (HIV) or acquired immunodeficiency syndrome (AIDS)-related illness.
Active infection requiring therapy (except nail fungus), positive tests for Hepatitis B surface antigen (HBsAg) with detected Hepatitis B virus (HBV) DNA or positive Hepatitis C antibody with detected Hepatitis C virus (HCV) RNA.
Prior gastrectomy or upper bowel removal or any other gastrointestinal disorder or defect e.g., malabsorption disorder such as Crohn's disease or ulcerative colitis, that would interfere with absorption of Compound 1.
Patients who are receiving treatment with medications that cannot be discontinued prior to study entry and that are considered to be any of the following
   known to be strong inducers or inhibitors of CYP3A4/5
   known to be substrates of CYP3A4/5, OAT3, OATP1B1, and MATE1/2-K with a narrow therapeutic index
Females who are pregnant or breastfeeding:
   Women of childbearing potential must not be considering getting pregnant during the study.
   Patients of reproductive potential (male & female) must practice an effective method of contraception during treatment and for 30 days following the last dose of Compound 1. Patients unwilling to do so will be excluded.
Impaired cardiac function or clinically significant cardiac diseases, including any of the following:
   History or presence of ventricular tachyarrhythmia
   Presence of unstable atrial fibrillation (ventricular response >100 BPM); patients with stable atrial fibrillation are eligible, provided they do not meet any of the other cardiac exclusion criteria
   Angina pectoris or acute myocardial infarction ≤6 months prior to starting study drug
   Other clinically significant heart disease (e.g., symptomatic congestive heart failure; uncontrolled arrhythmia or hypertension; history of labile hypertension or poor compliance with an antihypertensive regimen)
   Patients with a drug eluting stent for cardiovascular purposes placed 6 months prior to starting study drug
   Corrected QT interval using Fridericia's method (QTcF)>480 msec on baseline ECG (mean of baseline values). If electrolytes are abnormal, they may be corrected, and baseline ECGs should be repeated.
Known to have previously received Compound 1
Presence of any other condition that may increase the risk associated with study participation or may interfere with the interpretation of study results and, in the opinion of the investigator, would make the patient inappropriate for entry into the study.

### Example 2. Treatment with Compound 1 Reduces Ocular Tumor Size

A patient (patient 1) (enrolled in NCT05187884) was treated with Compound 1 (300 mg BID) for 27 days, prior to potential planned interventional procedure comprising enucleation. A 22% ocular tumor reduction in maximal apical height by ocular ultrasound (after about 28 days administration of Compound 1) was observed prior to the planned interventional procedure. The patient received planned definitive therapy of enucleation and subsequently received 6 months of adjuvant Compound 1 therapy for 6 months.

A patient (patient 2) (enrolled in NCT02601378) with metastatic disease to liver and peritoneum at the time of diagnosis, and with primary lesion intact (intact ocular tumor) was treated with Compound 1 (300 mg BID). PET scans (after about two weeks administration of Compound 1) showed a 74% reduction in the metabolic activity of the ocular tumor (reduction in FDG (Fluorodeoxyglucose) avidity) as measured by Standard Uptake Value (SUV). The patient reported improvement in visual symptoms in affected eye.

A patient (patient 3) (enrolled in NCT05187884) was treated with Compound 1 (300 mg BID) for about 28 days and continues on treatment, prior to potential planned interventional procedure comprising enucleation. A ˜10% ocular tumor reduction in maximal apical height by ocular ultrasound (after 28 days administration of Compound 1) was observed prior to the planned interventional procedure.

A patient (patient 4) (enrolled in NCT05187884) was treated with Compound 1 (300 mg BID) for about 28 days, prior to the planned interventional procedure (e.g., enucleation). A 31% ocular tumor reduction in maximal apical height by ocular ultrasound was observed (after about 28 days administration of Compound 1). PET scans (after about one month of administering Compound 1) showed a 42% reduction in the metabolic activity of the ocular tumor (reduction in FDG (Fluorodeoxyglucose) avidity) as measured by Standard Uptake Value (SUV).

A patient (patient 5) (enrolled in NCT05187884) was treated with Compound 1 (300 mg BID) for about 28 days. A 13% ocular tumor reduction in maximal apical height by ocular ultrasound was observed (after about 28 days administration of Compound 1). The patient is continuing on treatment to maximal benefit up to 6 months prior to planned enucleation. The patient was continuing treatment with Compound 1 (300 mg BID) for additional about 3 months (in addition to about 28 days as described above), and a 24% ocular tumor reduction in maximal apical height by ocular ultrasound was observed. The reduction in tumor size of the patient enabled plaque brachytherapy as a primary interventional treatment rather than originally planned enucleation and the eye was preserved. The patient continues on adjuvant therapy for potentially up to 6 months.

A patient (patient 6) (enrolled in NCT05187884) was treated with Compound 1 (300 mg BID) for about 56 days, prior to the planned interventional procedure (e.g. enucleation). A 11% ocular tumor reduction in maximal apical height and 23% in Largest Basal Diameter by ocular ultrasound was observed. The patient is continuing on treatment to maximal benefit up to 6 months prior to planned enucleation. The patient continued treatment with Compound 1 (300 mg BID) for about 2 months (in addition to the 56 days as described above), and a 24% ocular tumor reduction in maximal apical height by ocular ultrasound was observed. The patient continued treatment with Compound 1 (300 mg BID) for about 1 month (in addition to about 4 months described above), and a 45% ocular tumor reduction in maximal apical height and 63% reduction in Largest Basal Diameter by ocular ultrasound was observed. The reduction in tumor size of the patient enabled plaque brachytherapy as a primary interventional treatment rather than originally planned enucleation and the eye was preserved.

A patient (patient 7) (enrolled in NCT05187884) was treated with Compound 1 (300 mg BID) for about 84 days, prior to potential planned interventional procedure comprising enucleation. A 14.3% ocular tumor reduction in maximal apical height and 15.2% reduction in Largest Basal Diameter by ocular ultrasound (after about 84 days administration of Compound 1) was observed prior to the planned interventional procedure.

A patient (patient 8) (enrolled in NCT05187884) was treated with Compound 1 (300 mg BID) for about 84 days and continues on treatment, prior to potential planned interventional procedure comprising enucleation. A 28.5% ocular tumor reduction in maximal apical height and 19.4% reduction in Largest Basal Diameter by ocular ultrasound (after about 84 days administration of Compound 1) was observed prior to the planned interventional procedure.

A patient (patient 9) (enrolled in NCT05187884) was treated with Compound 1 (300 mg BID) for about 56 days and continues on treatment, prior to potential planned interventional procedure comprising enucleation. A 4.0% ocular tumor reduction in maximal apical height and 2.1% reduction in Largest Basal Diameter by ocular ultrasound (after about 56 days administration of Compound 1) was observed prior to the planned interventional procedure.

All ocular lesion reduction measurements were based on baseline ocular lesion, which is the ocular tumor measurements before any treatment with Compound 1

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method of treating uveal melanoma in a patient having an ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure.

2. The method of claim 1, wherein the ocular tumor is defined by a set of dimensions comprising (i) greater than or equal to 5 mm largest basal basal diameter (LBD) and any apical height, or (ii) any LBD and greater than 3 mm apical height.

3. A method of treating a patient having uveal melanoma with an ocular tumor with a size indicated for an interventional procedure comprising enucleation of the eye, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

4. The method of claim 3, wherein the ocular tumor is defined by a set of dimensions comprising (i) up to 22 mm in LBD and/or (ii) up to 15 mm in apical height.

5. A method of treating a patient having uveal melanoma with an ocular tumor with a size indicated for an interventional procedure comprising irradiating the ocular tumor, the method comprising
a) administering a PKC inhibitor to the patient as a neoadjuvant therapy;
b) reducing the size of the ocular tumor by at least 10%; and
c) treating the reduced-size ocular tumor with an interventional procedure other than enucleation.

6. The method of claim 5, wherein the ocular tumor is defined by a set of dimensions comprising (i) at least 6 mm in LBD and/or (ii) at least 3 mm in apical height.

7. The method of claim 1, wherein the interventional procedure comprises local surgical resection of the tumor, irradiating the tumor, or any combination thereof.

8. The method of claim 1, wherein the interventional procedure comprises irradiating the tumor.

9. A method of treating uveal melanoma in a patient having an ocular tumor, the method comprising:
a) determining a first therapeutically effective dose of radiation for treating the ocular tumor;
b) administering a PKC inhibitor to the patient as a neoadjuvant therapy;
c) reducing the size of the ocular tumor; and
d) irradiating the reduced-size ocular tumor with a second therapeutically effective dose of radiation less than the first therapeutically effective dose of radiation.

10. The method of claim 9, wherein in step (c) the ocular tumor is reduced by at least 10%.

11. The method of claim 9, wherein the ocular tumor before step (a) is defined by a set of dimensions comprising (i) at least 6 mm in LBD and/or (ii) at least 3 mm in apical height.

12. The method of claim 1, wherein in step (d) the second therapeutically effective dose of radiation is at least 10% less than the first therapeutically effective dose of radiation.

13. The method of claim 1, wherein the PKC inhibitor is Compound 1: 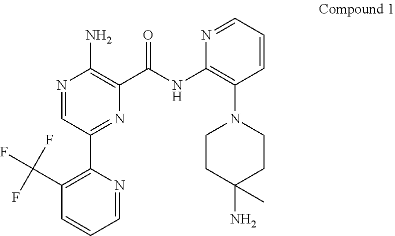 [SMILES: CC1(N)CCN(c2cccnc2NC(=O)c2nc(-c3ncccc3C(F)(F)F)cnc2N)CC1]
or a pharmaceutically acceptable salt thereof.

14. The method of claim 1, wherein the interventional procedure is a globe preserving treatment (GPT).

15. The method of claim 1, wherein the interventional procedure is selected from the group consisting of plaque brachytherapy (PBT), external beam radiotherapy (EBRT), stereotactic radiosurgery (SRS), and any combination thereof.

16. The method of claim 1, wherein the interventional procedure is plaque brachytherapy (PBT).

17. The method of claim 1, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

18. The method of claim 1, wherein the method further comprises continuing administering a PKC inhibitor to the patient as an adjuvant therapy during the period of the interventional procedure.

19. The method of claim 1, wherein the method further comprises continuing administering a PKC inhibitor as an adjuvant therapy after the completion of the interventional procedure.

20. The method of claim 19, wherein the adjuvant therapy is administered at least 4 weeks, at least 5 weeks, or at least 6 weeks after the completion of the interventional procedure.

21. The method of claim 1, wherein the PKC inhibitor is administered orally.

22. The method of claim 1, wherein the method further comprises preserving the eye globe which had the ocular tumor.

23. The method of claim 1, wherein the method further comprises preserving a function of the eye which had the ocular tumor.

24. The method of claim 23, wherein preserving the function of the eye comprises at least partially preserving the eye function.

25. The method of claim 1, wherein the method further comprises preventing irreversible vision loss.

26. The method of claim 1, wherein the method further comprises preserving visual symptoms.

27. The method of claim 1, wherein the method further comprises preserving depth perception.

28. The method of claim 1, wherein the method further comprises preserving vision for greater than at least 1 year.

29. The method of claim 1, wherein the method further comprises preventing metastasis.

30. The method of claim 1, wherein the method further comprises delaying onset of metastasis.

31. The method of claim 1, wherein the ocular tumor is spatially separated from the optic nerve.

32. The method of claim 1, wherein the ocular tumor, prior to administering the neoadjuvant therapy, has a size
i) from 5 mm to 19 mm LBD and less than 2.5 mm apical height,
ii) less than or equal to 19 mm LBD and 2.5-10 mm apical height,
iii) greater than 19 mm LBD,
iv) greater than 10 mm apical height and any size LBD,
v) greater than 8 mm apical height and the tumor is located close to the optic nerve,
vi) greater than 6 mm and less than 12 mm apical height, and not greater than 16 mm LBD,
vii) greater than 3 mm and less than 8 mm apical height, and not greater than 16 mm LBD,
viii) greater than 8 mm apical height, and greater than 16 mm LBD,
ix) not greater than 16 mm LBD, or
x) greater than 10 mm in apical height.

33. The method of claim 1, wherein the size of the ocular tumor after administering the neoadjuvant therapy but prior to the interventional procedure is reduced by at least 20%.

34. The method of claim 1, wherein the PKC inhibitor, or an equivalent dose of a pharmaceutically acceptable salt thereof, is administered at a dose of about 200 mg to about 400 mg two times per day (BID).

35. The method of claim 1, wherein the PKC inhibitor, or an equivalent dose of a pharmaceutically acceptable salt thereof, is administered at a dose of about 300 mg two times per day (BID).

36. The method of claim 1, wherein the PKC inhibitor, or an equivalent dose of a pharmaceutically acceptable salt thereof, is administered at a dose of about 400 mg to about 600 mg daily.

37. The method of claim 1, wherein the PKC inhibitor, or an equivalent dose of a pharmaceutically acceptable salt thereof is administered daily for at least 7 days, at least 14 days, at least 21, days, at least 28 days, at least 56 days, at least 84 days, at least, 112 days, at last 140 days, or at least 168 days.

38. The method of claim 1, wherein the uveal melanoma is a solid tumor harboring GNAQ or GNA11 mutations.

39. The method of claim 1, wherein the ocular tumor is intraocular tumor.

40. The method of claim 1, wherein the size of the ocular tumor is measured using RECIST 1.1 criteria.

41. The method of claim 1, wherein the size of the ocular tumor is determined by CT scan, MRI, or ultrasound, or a combination thereof.

42. The method of claim 1, wherein the neoadjuvant therapy is administered to the patient for at least 1 month, at least 2 months, at least 3 months, at least 4 months, at least 5 months, or at least 6 months.

43. The method of claim 1, wherein the adjuvant therapy is administered to the patient for at least 1 month, at least 2 months, at least 3 months, at least 4 months, at least 5 months, or at least 6 months.
